SEQUENCE LISTING (1 sequence)

SEQ ID NO: 1 - protein, 14 aa, Artificial Sequence
  Completely Synthesized
SGSGPRPLRRESEI

(19) United States
(12) Patent Application Publication
(10) Pub. No.: US 2007/0099254 A1
(43) Pub. Date: May 3, 2007

(54) Polypeptide arrays, methods of polypeptide screening, and mass spectrometric analysis of polypeptides

(76) Inventors: Viorica Lopez Avila, Sunnyvale, CA (US); David L. Hirschberg, Menlo Park, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/500,111

(22) Filed: Aug. 7, 2006

Related U.S. Application Data
(60) Provisional application No. 60/731,709, filed on Oct. 31, 2005.

(51) Int. Cl. C12Q 1/34

(52) U.S. Cl. 435/18

(57) ABSTRACT

Embodiments of the present disclosure provide screening methods for identifying target molecules capable of being post-translationally modified.

### CROSS-REFERENCE TO RELATED APPLICATION

This application claims priority to copending U.S. provisional application entitled, “Polypeptide Arrays, Methods of Polypeptide Screening, and Mass Spectrometric Analysis of Polypeptides,” having Ser. No. 60/731,709, filed Oct. 31, 2005, which is entirely incorporated herein by reference.

### BACKGROUND

Post-translational modification of proteins, namely, phosphorylation, dephosphorylation, sulfation, and desulfation of proteins, are important processes in regard to many diseases and disorders. Therefore, much effort has been made in determining which proteins or portions thereof are implicated in these processes.

Protein phosphorylation is a common regulatory mechanism used by cells to selectively modify proteins carrying regulatory signals from outside the cell to the nucleus. The proteins that execute these biochemical modifications are a group of enzymes known as protein kinases. Kinases represent a large family of enzymes with an estimated 1000 different forms present in the human genome. They play an important role in regulating signal transduction by adding phosphate groups to various proteins in cellular signaling pathways. Depending on the phosphate-accepting protein, the addition of a phosphate group may up-regulate or down regulate the signal. Protein kinases come in two general classes, which are defined by the particular amino acid that is phosphorylated. These two classes are tyrosine kinases and serine/threonine kinases.

The complementary activity of dephosphorylation is a function of another group of enzymes known as protein phosphatases. Protein phosphatases can be broadly classified into two families, protein serine/threonine (ser/thr) phosphatases (PP) and protein tyrosine (tyr) phosphatases (PTPase). Tyrosine phosphatases generally catalyze the dephosphorylation of tyrosine residues, though some have dual specificity for both phosphoser/thr and phospho/tyr. Ser/thr phosphatases catalyze the dephosphorylation of phosphoserine and phosphothreonine residues in proteins and peptides and have been further divided into three types, PP1, PP2A, and PP2B. PTP/PPases provide reversible protein phosphorylation through the dephosphorylation of phosphoamino acids and function in the control of cellular proliferation, differentiation and other cellular processes. These phosphatases can be further classified into soluble or transmembrane proteins.

Kinases and phosphatases are often validated drug targets since human diseases are frequently linked to dysregulation of cellular signaling pathways. The profound cellular effects mediated by tyrosine kinases and serine kinases have made them attractive targets for the development of new therapeutic molecules. For example, the cellular effects include their putative role in angiogenesis, lymphoid development, and insulin resistance, coupled with the implication that mutant or defective tyrosine kinase variants may be involved in tumorigenesis.

Sulfation is an important pathway in the metabolism of many neurotransmitters, hormones, drugs and other xenobiotics. Sulfate conjugation is catalyzed by members of a gene superfamily of cytosolic sulfotransferase enzymes.

Sulfotransferases are enzymes that catalyze the transfer of a sulfate from a donor compound to an acceptor compound, usually placing the sulfate moiety at a specific location on the acceptor compound. Sulfotransferases, present in most organisms and in all human tissues, mediate sulfation of different classes of acceptors for a variety of biological functions. To date, more than 30 sulfotransferase cDNAs have been isolated from animal, plant, and bacterial sources. The varied and important roles sulfotransferases play in biological systems have been uncovered, including detoxification, cell signaling, and modulation of receptor binding.

Sulfation functions in the metabolism of xenobiotic compounds, steroid biosynthesis, and modulating the biological activity, inactivation and elimination of potent endogenous chemicals such as thyroid-hormones, steroids and catechols. This process is reversible, involving the sulfotransferase enzymes that catalyze the sulfation and the sulfatases that hydrolyze the sulfate esters formed by the action of the sulfotransferases. Accordingly, the interplay between these families regulates the availability and biological activity of many xenobiotic and endogenous chemicals.

The presence of sulfated components depends upon the availability of key members of the sulfate pathway, i.e., substrates and activated sulfate donor molecules (co-substrates), and the balance between sulfation and sulfate conjugate hydrolysis depends upon the activity and localization of the sulfotransferases and the sulfatases. In general, a divalent sulfate is converted to adenosine 5′ phosphosulfate (PAPS) by hydrolysis of ATP. This compound is in turn converted to 3′ phosphoadenosine 5′ phosphosulfate by hydrolysis of ATP to ADP. This compound is then converted to adenosine 3′5′ biphosphate concurrently with the formation of 4-nitrophenolsulfate from 4-nitrophenol. An ARS then cleaves the monovalent sulfate from the 4-nitrophenolsulfate to produce the original 4-nitrophenol. This process forms the basis of the sulfation system in humans. Over- or under-production of any of these key molecules can result in sulfate-related disorders.

### SUMMARY

Embodiments of the present disclosure provide screening methods for identifying target molecules capable of being post-translationally modified. Briefly described, one embodiment of screening, among others, includes: providing a substrate having a sample-retaining structure disposed on the substrate and a post-translational modifying solution disposed in the retaining structure; providing a matrix assisted laser desorption/ionization (MALDI) plate having a plurality of target molecules disposed on the MALDI plate, wherein each target molecule has a known addressable position on the MALDI plate; exposing the plurality of target molecules to the post-translational modifying solution by disposing the MALDI plate onto the substrate and retaining structure so that the plurality of target molecules are disposed into the post-translational modifying solution, wherein the post-translational modifying solution reacts with select target molecules to produce post-translational modified target molecules; analyzing the post-translational modified target molecules using a matrix assisted laser desorption/ionization mass spectrometry system (MALDI-MS system).

Briefly described, another embodiment of screening, among others, includes: providing a substrate having a plurality of sample-retaining structures disposed on the substrate, wherein at least two sample-retaining structures have a different post-translational modifying solution disposed in each of the at least two sample-retaining structures; providing a plurality of supports having at least one target molecule disposed thereon; disposing a portion of the supports into each of the sample-retaining structures, wherein the target molecules are disposed into one of the post-translational modifying solutions in the sample-retaining structures, wherein the post-translational modifying solution reacts with select target molecules to produce post-translational modified target molecules; separating the supports from the post-translational modifying solutions; removing one or both of the target molecules or the post-translational modified target molecules from the supports; disposing one or both of the target molecules or the post-translational modified target molecules onto a matrix assisted laser desorption/ionization (MALDI) plate, wherein each target molecule or the post-translational modified target molecule has a known addressable position on the MALDI plate; operatively associating the MALDI plate with a matrix assisted laser desorption/ionization mass spectrometry system (MALDI-MS system); illuminating a portion of the MALDI plate with a laser of the MALDI-MS system, wherein the target molecules or post-translational modified target molecules are ionized; and collecting mass spectral data of the ionized target molecules or post-translational modified target molecules.

Other systems, methods, features, and advantages of the present disclosure will be or become apparent to one with skill in the art upon examination of the following drawings and detailed description. It is intended that all such additional systems, methods, features, and advantages be included within this description, be within the scope of the disclosure, and be protected by the accompanying claims.

### BRIEF DESCRIPTION OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a representative method of screening target molecules.

FIGS. 2A through 2C illustrate the introduction of target molecules to a post-translational modifying solution.

FIG. 3 illustrates another representative method of screening target molecules.

FIGS. 4A through 4C illustrates another representative method of screening a target molecule against a plurality of post-translational modifying solutions.

FIG. 5 is a block diagram that illustrates a representative schematic diagram of an MALDI-MS system.

FIG. 6 illustrates a MALDI mass spectrum of biotinylated Kir 2.1 peptide (biotin- SEQ ID: 1 (SGSGPRPLRRESEI)-COOH)).

FIG. 7 illustrates a MALDI mass spectrum of biotinylated and phosphorylated Kir2. 1 peptide, where only difference in the sequence in that the serine (third to last amino acid) is phosphorylated (biotin-SEQ ID: 1 (SGSGPRPLRRES(Phosphate)EI)-COOH).

FIG. 8 illustrates a peptide array that contains 181 peptides, each printed in quadruplicate (32×48 spots).

### DETAILED DESCRIPTION

Embodiments of the present disclosure will employ, unless otherwise indicated, techniques of synthetic organic chemistry, biochemistry, molecular biology, and the like, that is within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

“Polypeptide” refers to peptides, proteins, glycoproteins, portions of each, and the like. “Polypeptide” refers to two or more amino acids joined to each other by peptide bonds or modified peptide bonds, (i.e., peptide isosteres). “Polypeptide” refers to both short chains, commonly referred to as peptides, oligopeptides, or oligomers, and to longer chains, generally referred to as proteins. “Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

As used herein, polynucleotides include single or multiple stranded configurations, where one or more of the strands may or may not be completely aligned with one another. The terms “polynucleotide” and “oligonucleotide” shall be generic to polydeoxynucleotides (containing 2-deoxy-D-ribose), to polyribonucleotides (containing D-ribose), to any other type of polynucleotide which is a glycoside of a purine or pyrimidine base, and to other polymers in which the conventional backbone has been replaced with a non-naturally occurring or synthetic backbone or in which one or more of the conventional bases has been replaced with a non-naturally occurring or synthetic base. The terms “ribonucleic acid” and “RNA” as used herein mean a polymer composed of ribonucleotides. The terms “deoxyribonucleic acid” and “DNA” as used herein mean a polymer composed of deoxyribonucleotides.

A “nucleotide” refers to a sub-unit of a nucleic acid (whether DNA or RNA or an analogue thereof), which includes a phosphate group, a sugar group and a nitrogen containing base, as well as analogs of such sub-units.

A “nucleoside” references a nucleic acid subunit including a sugar group and a nitrogen containing base. It should be noted that the term “nucleotide” is used herein to describe embodiments of the disclosure, but that one skilled in the art would understand that the term “nucleoside” and “nucleotide” are interchangable in most instances. One skilled in the art would have the understanding that additional modification to the nucleoside may be necessary, and one skilled in the art has the knowledge to perform such modification.

The term “sample” as used herein relates to a material or mixture of materials, typically, although not necessarily, in fluid form, containing one or more components of interest.

The term “array” encompasses the term “microarray” and refers to an ordered array presented for binding to nucleic acids and the like.

An “array,” includes any two-dimensional or substantially two-dimensional (as well as a three-dimensional) arrangement of addressable regions including nucleic acids (e.g., particularly oligonucleotides or synthetic mimetics thereof) and the like.

Where the arrays are arrays of nucleic acids, the nucleic acids may be adsorbed, physisorbed, chemisorbed, and/or covalently attached to the arrays at any point or points along the nucleic acid chain.

The substrate may carry one, two, four or more arrays disposed on a front surface of the substrate. Depending upon the use, any or all of the arrays may be the same or different from one another, and each may contain multiple spots or features. A typical array may contain one or more features, including more than two, more than ten, more than one hundred, more than one thousand, more ten thousand, or even more than one hundred thousand features, in an area of less than about 20 cm or even less than about 10 cm² (e.g., less than about 5 cm², including less than about 1 cm² or less than about 1 MM2 (e.g., about 100 μm², or even smaller)). For example, features may have widths (that is, diameter, for a round spot) in the range from about 10 μm to 1.0 cm. In other embodiments each feature may have a width in the range of about 1.0 μm to 1.0 mm, about 5.0 μm to 500 μm, and about 10 μm to 200 μm. Non-round features may have area ranges equivalent to that of circular features with the foregoing width (diameter) ranges. At least some, or all, of the features are of different compositions (for example, when any repeats of each feature composition are excluded, the remaining features may account for at least about 5%, about 10%, about 20%, about 50%, about 95%, about 99% or about 100% of the total number of features). Inter-feature areas may typically (but not essentially) be present, which do not carry any nucleic acids (or other biopolymer or chemical moiety of a type of which the features are composed). Such inter-feature areas typically may be present where the arrays are formed by processes involving drop deposition of reagents but may not be present when, for example, light directed array fabrication processes are used. It will be appreciated, though, that the inter-feature areas, when present, could be of various sizes and configurations.

Each array may cover an area of less than about 200 cm², or even less than about 50 cm², about 5 cm², about 1 cm², about 0.5 cm², or about 0.1 cm². In certain embodiments, the substrate carrying the one or more arrays may be shaped generally as a rectangular solid (although other shapes are possible), having a length of more than about 4 mm and less than about 150 mm, usually more than about 4 mm and less than 80 mm, more usually less than 20 mm; a width of more than about 4 mm and less than about 150 mm, usually less than 80 mm and more usually less than 20 mm; and a thickness of more than about 0.01 mm and less than about 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally, in this situation, the substrate may be relatively transparent to reduce the absorption of the incident illuminating laser light and subsequent heating if the focused laser beam travels too slowly over a region. For example, the substrate may transmit at least about 20% or 50% (or even at least about 70%, about 90%, or about 95%) of the illuminating light incident on the front of the substrate, as may be measured across the entire integrated spectrum of such illuminating light or, alternatively, at about 532 nm or 633 nm.

Arrays can be fabricated using drop deposition from pulse-jets of either nucleic acid precursor units (such as monomers), as in the case of in situ fabrication, or of a previously obtained nucleic acid. Such methods are described in detail in, for example, U.S. Pat. No. 6,242,266, U.S. Pat. No. 6,232,072, U.S. Pat. No. 6,180,351, U.S. Pat. No. 6,171,797, and U.S. Pat No. 6,323,043. As already mentioned, these references are incorporated herein by reference.

An array “package” may be the array plus only a substrate on which the array is deposited, although the package may include other features (such as a housing with a chamber). A “chamber” references an enclosed volume (although a chamber may be accessible through one or more ports). It will also be appreciated that, throughout the present application, words such as “top,” “upper,” and “lower” are used in a relative sense only.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of the present application, the “probe” will be referenced in certain embodiments as a moiety in a mobile phase (typically fluid), to be detected by “targets”, which are bound to the substrate at the various regions.

A “scan region” refers to a contiguous (preferably rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For example, in fluorescent detection embodiments, the scan region includes the entire area of the slide scanned in each pass of the lens, between the first feature of interest and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

As used herein with regard to post-translational modification of a polypeptide, the term “site” refers to an amino acid or amino acid sequence of a natural binding domain or binding partner that is recognized by a post-translational modification compound for the purpose of post-translational modification (e.g., addition or removal of a phosphate moiety) of the polypeptide or a portion thereof. It is contemplated that a site comprises a small number of amino acids, including as few as one, more typically from 1 to about 10, and less often up to about 30 amino acids. It is further contemplated that a site comprises fewer than the total number of amino acids present in the polypeptide.

As used herein, “post-translational modification”, “post-translational modifying”, and the like, identify post-translational modifications such as, but not limited to, phosphorylation, sulfation, and/or glycosylation.

As used herein, “phosphorylation” and “dephosphorylation” refer to the addition or removal of a phosphate moiety to/from a polypeptide, respectively.

As used herein, “sulfation” and “desulfation” refer to the addition or removal of a sulfate moiety to/from a polypeptide, respectively.

As used herein, the term “moiety” refers to a post-translationally added or removed phosphate group or sulfate group; the terms “moiety” and “group” are used interchangeably.

The term “screening” refers to the identification of one or more target proteins, for instance, from among large collections of candidate proteins.

It should be noted that ratios, concentrations, amounts, and other numerical data may be expressed herein in a range format. It is to be understood that such a range format is used for convenience and brevity and, thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also to include the individual concentrations (e.g., 1%, 2%, 3%, and 4%) and the sub-ranges (e.g., 0.5%, 1.1%, 2.2%, 3.3%, and 4.4%) within the indicated range.

All patents, patent applications, and publications mentioned herein are hereby incorporated by reference in their entireties.

Discussion

Embodiments of the present disclosure provide for polypeptide analysis, methods of screening polypeptides, and mass spectrometric analysis of polypeptides. In particular, the present disclosure describes methods of screening a plurality of polypeptides (e.g., proteins or portions thereof) using a single post-translational modifying solution (e.g., a solution including kinases and ATP), and methods of screening one polypeptide using a plurality of post-translational modifying solutions. The polypeptides can be attached (e.g., prior to a post-translational modification treatment or after a post-translational modification treatment) to a substrate such as a matrix assisted laser desorption/ionization (MALDI) plate, and analyzed. The polypeptides, some of which may have been post-translational modified, can be analyzed using a mass spectrometry system, such as a MALDI mass spectrometry system. In other embodiments, the polypeptide can be replaced with a polynucleotide and reacted with appropriate reactants to achieve the intended goal that would be within the knowledge of one of skill in the art. However, the remainder of this disclosure is described in reference to polypeptides.

Embodiments of this disclosure overcome at least some deficiencies in other methods. Namely, antibodies or other identifying agents (e.g., fluorescent tags) are not necessary to identify the target polypeptides.

As mentioned above, the methods of this disclosure can be used to identify target molecules (e.g., polypeptides, proteins, or portions thereof) after exposure to a post-translational modifying solution to determine if the target molecule was post-translationally modified. The post-translational modifications to be detected include, but are not limited to, phosphorylation, dephosphorylation, sulfation, desulfation, and glycosylation. The post-translational modifying solution can include, but is not limited to, kinases (i.e., phosphorylation), phosphatases (i.e., dephosphorylation), sulfotransferases (i.e., sulfation), and sulfatases (i.e., desulfation). The post-translational modifying solution may also include other components that enable the post-translational modification to occur.

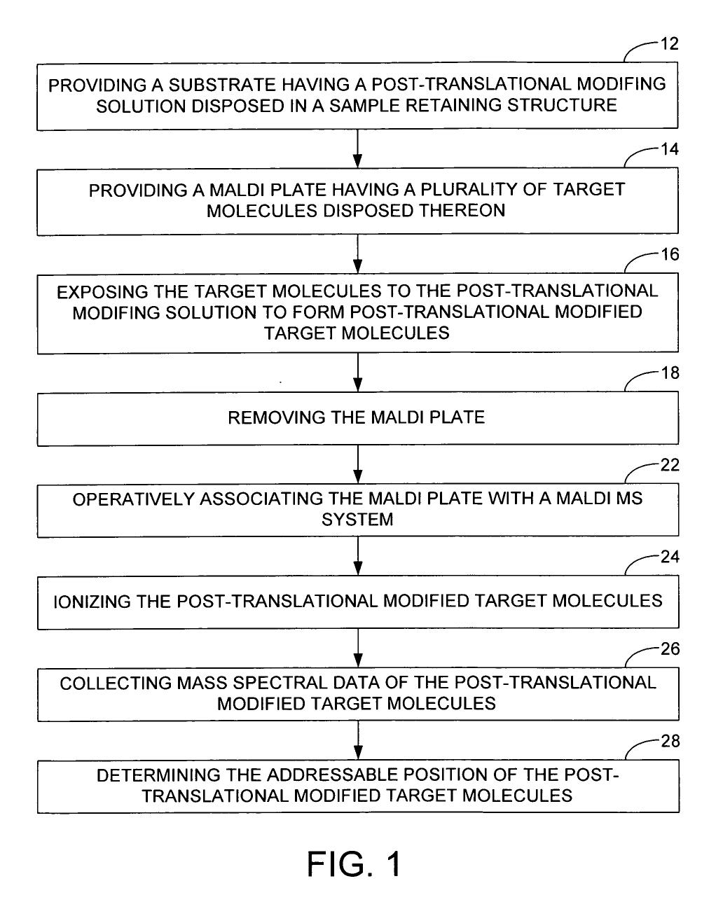

FIG. 1 illustrates a representative method of screening target molecules (e.g., polypeptides). In Block 12, a substrate having a post-translational modifying solution disposed in a sample-retaining structure disposed on the substrate is provided. Details regarding the sample-retaining structure are described in more detail below. In Block 14, a MALDI plate having a plurality of target molecules disposed thereon is provided.

The MALDI plate is typically configured to be used in a MALDI mass spectrometry system (e.g., capable of being operatively associated or inserted or otherwise coupled to a MALDI mass spectrometry system). The MALDI plate can be described as an array of addressable polypeptides (or other target molecules) or can be described as having a plurality of arrays of addressable polypeptides.

In the latter embodiment, the substrate could include a plurality of sample-retaining structures each having the same or different post-translational modifying solutions. If a plurality of post-translational modifying solutions are disposed in different sample-retaining structures, then a plurality of different polypeptides can be assayed against a plurality of different post-translational modifying solutions on a MALDI plate.

The target molecules can include a plurality of polypeptide sequences that correspond to one or more proteins or portions of proteins. For example, the MALDI plate may include a library of polypeptide sequences corresponding to proteins that are related to one or more disorders or diseases. The number and selection of the polypeptides to be included on the MALDI plate(s) depends, in part, upon the objective of the analysis, and, as such, is determined by one skilled in the art.

The post-translational modifying solution can include post-translational modifying molecules that may cause a post-translational modification in select target molecules. In one embodiment, a plurality of target molecules is analyzed against a single type of post-translational modifying molecule. For example, a plurality of polypeptides could be analyzed against a single kinase to test for phosphorylation.

As mentioned above, the post-translational modifying solution can include, but is not limited to, kinases for phosphorylation, phosphatases for dephosphorylation, sulfotransferases for sulfation, sulfatases for desulfation, and the like. The types of kinases, phosphatases, sulfotransferases, sulfatases, and other post-translational modifying molecules can include those known in the art or those found in the future.

The post-translational modifying solution can also include compounds used in conjunction with the post-translational modifying molecules to cause post-translational modification. The other compounds are known in the art. For example, a post-translational modifying solution including kinase also includes ATP. In another example, for sulfation a dilution buffer (e.g., 40 mM Pipes pH 6.8, 300 mM NaCl, 20 mM manganese chloride, 50 mM NaF, 1% Triton X-100 and 1 mM 5′-AMP), sulfation co-substrate (e.g., PAPS-3-phosphoadenosine-5-phosphosulfate 400 uM), and sulfotransferase (e.g., TPST-1 (tyrosyl protein sulfotransferase)), can be used. In still another example, for dephosphorylation, 25 mM ammonium bicarbonate and alkaline phosphatase 100 milliunits /uL, can be used. In still another embodiment, for desulfation an arylsulfatase in an acetate buffer at or near room temperature can be used.

The concentration of post-translational modifying molecules in the sample-retaining structure is in a range to cause post-translational modification of select target molecules. The concentration depends on factors that can be determined on a case-by-case basis.

In Block 16, the target molecules are exposed to the post-translational modifying solution. In particular, the MALDI plate is placed onto the substrate and sample-retaining structure so that the target molecules are disposed in the post-translational modifying solution. In some cases, the MALDI plate may include one or more target molecules (select target molecules) that undergo post-translational modification in the presence of the post-translational modifying molecules. For example, a select molecule may react with a kinase to phosphorylize the select molecule in one or more sites.

The target molecules and the post-translational modifying solution can be incubated in conditions under which post-translational modification may occur. The conditions (e.g., time and temperature) can vary depending, in part, on the polypeptides, post-translational modifying solution, and the like.

In Block 18, the MALDI plate is removed from the substrate and sample-retaining structure after incubation.

In Block 22, the MALDI plate is operatively associated with a MALDI mass spectrometry system. Details regarding MALDI mass spectrometry systems are discussed below.

In Block 24, the target molecules and the post-translational modified molecules, if any, are ionized using the MALDI mass spectrometry system. In Block 26, mass spectral data of the ionized target molecules and the post-translational modified molecules are collected. In Block 28, the target molecules that were post-transitionally modified can be determined by relating the target molecules that were post-translationally modified to the addressable position of each target molecule on the MALDI plate based on the MALDI mass spectral data. Analysis of mass spectral data and relating that information to the position on a MALDI plate (i.e., source of the ions) are known in the art.

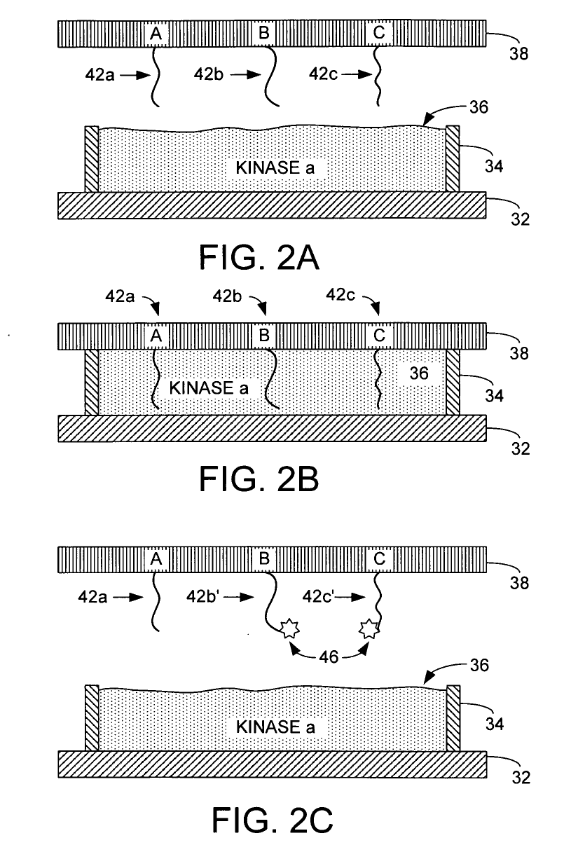

FIGS. 2A through 2C illustrate the introduction of target molecules to a post-translational modifying solution. FIG. 2A illustrates a substrate 32 having a sample-retaining structure 34 disposed thereon. The sample-retaining structure 34 includes a post-translational modifying solution 36 that includes kinase a. In addition, FIG. 2A illustrates a MALDI plate 38 that includes a plurality of target molecules A (42a), B (42b), and C (42c).

FIG. 2B illustrates the introduction and incubation of the target molecules A (42a), B (42b), and C (42c) in the kinase a containing, post-translational modifying solution 36. The MALDI plate 38 forms a fluid tight seal with the sample-retaining structure 34. The target molecules A (42a), B (42b), and C (42c) can be incubated for an appropriate amount of time to achieve post-translational modification, if it would occur.

FIG. 2C illustrates the removal of the MALDI plate 38 from the sample-retaining structure 34. Target molecule A (42a) was not phosphorylated by the kinase a, while target molecules B (42b) and C (42c) were phosphorylated (as shown by phosphate 46) by kinase a.

After appropriate post incubation treatment (e.g., washing), the MALDI plate can be analyzed using a MALDI mass spectrometry system, for example. Mass spectral data can be obtained for each of the addressable positions on the MALDI plate. The mass spectral data indicates which, if any, of the plurality of the target molecules is post-translationally modified. In FIGS. 2A through 2B, the mass spectral data would show that target molecules B and C were phosphorylated by the kinase a.

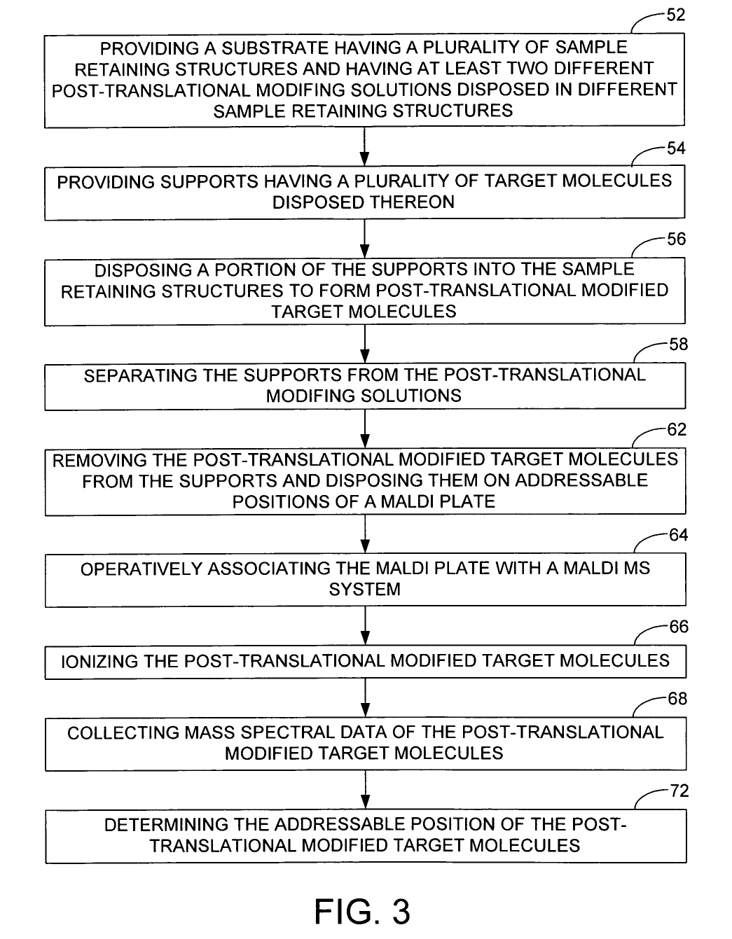

FIG. 3 illustrates another representative method of screening target molecules (e.g., polypeptides). In Block 52, a substrate having a plurality of sample-retaining structures is provided. Each sample-retaining structure includes a post-translational modifying solution, and in each case the post-translational modifying solution is different. In another embodiment, the substrate may include sample-retaining structures having the same post-translational modifying solution, but at least two of the post-translational modifying solutions are different. Details regarding the sample-retaining structure are described in more detail below. In Block 54, a plurality of supports having a target molecule disposed thereon is provided. The target molecule can include a polypeptide sequence that corresponds to a protein.

The post-translational modifying solution can include post-translational modifying molecules that may cause a post-translational modification in select target molecules. In this embodiment, a single target molecule is analyzed against a plurality of types of post-translational modifying molecules. For example, a single polypeptide could be analyzed against a number of kinases to test for phosphorylation.

As mentioned above, the post-translational modifying solution can include, but is not limited to, kinases for phosphorylation, phosphatases for dephosphorylation, sulfotransferases for sulfation, sulfatases for desulfation, and the like. The types of kinases, phosphatases, sulfotransferases, sulfatases, and other post-translational modified molecules can include those known in the art or those found in the future.

As mentioned above, the post-translational modifying solution can also include compounds used in conjunction with the post-translational modifying molecules to cause post-translational modification.

The concentration of post-translational modifying molecules in the sample-retaining structure is in a range to cause post-transitional modification of select target molecules. The concentration depends on factors that can be determined on a case-by-case basis.

In Block 56, the target molecule is exposed to the plurality of post-translational modifying solutions in different sample-retaining structures. In particular, the supports are placed into each of the sample-retaining structures so that the target molecules are disposed into each of the post-translational modifying solutions. The target molecule may undergo post-translational modification in the presence of one or more of the post-translational modifying molecules. For example, a target molecule may react with one or more kinases to phosphorylize the target molecule on one or more sites.

The target molecule and the post-translational modifying solutions can be incubated in conditions under which post-translational modification may occur. The conditions (e.g., time and temperature) can vary depending, in part, on the polypeptides, post-translational modifying solution, and the like.

In Block 58, the supports are separated from the post-translational modifying solutions. The supports can be separated using a filtration system included in the substrate sample-retaining structures. Alternatively, the supports can be removed from the sample-retaining structures.

In Block 62, the target molecules and the post-translational modified target molecules are removed (e.g., unbound in whole or in part) from the supports. The target molecules and the post-translational modified target molecules can be removed by digestion methods or other methods depending on the amino acid sequence and the bonds to the supports. The target molecules and the post-translational modified target molecules (or portions thereof) are addressably positioned on a MALDI plate, for example.

In Block 64, the MALDI plate is operatively associated with a MALDI mass spectrometry system. Details regarding MALDI mass spectrometry systems are discussed below.

In Block 66, the target molecules and the post-translational modified molecules, if any, are ionized using the MALDI mass spectrometry system. In Block 68, mass spectral data of the ionized target molecules and the post-translational modified molecules are collected. In Block 72, the target molecules that were post-transitionally modified can be determined by relating the target molecules that were post-translationally modified to the addressable position of each target molecule on the MALDI plate based on the MALDI mass spectral data. Analysis of mass spectral data and relating that information to the position on a MALDI plate (i.e., source of the ions) are known in the art.

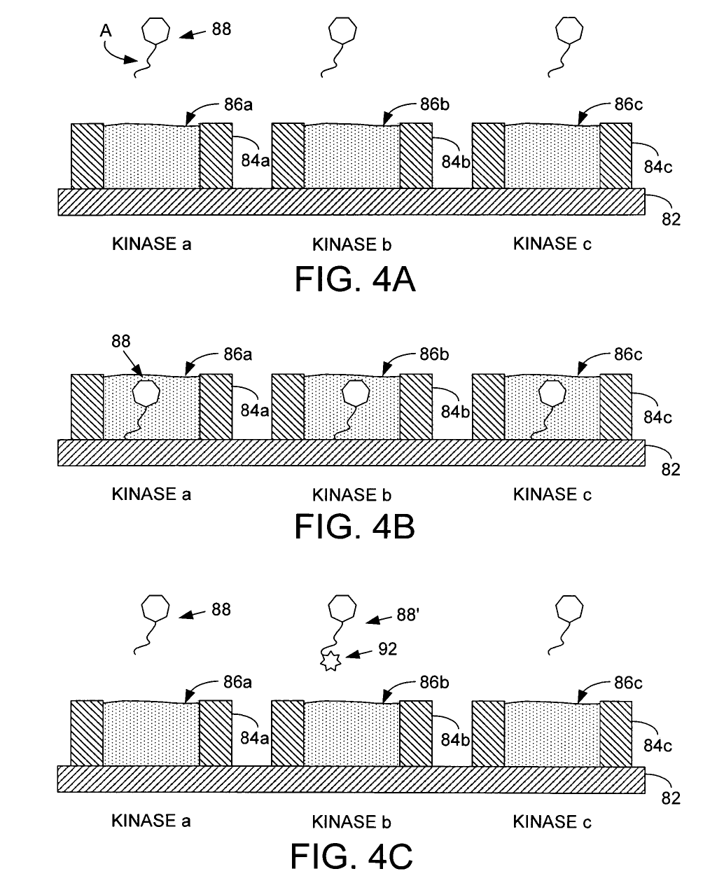

FIGS. 4A through 4C illustrate another representative method of screening a target molecule against a plurality of post-translational modifying solutions. FIG. 4A illustrates a substrate 82 having a plurality of sample-retaining structures 84a through 84c. Each sample-retaining structure includes three post-translational modifying solutions, kinases a, b, and c (86a through 86c). In addition, FIG. 4A illustrates a plurality of supports 88 having a target molecule disposed thereon. The target molecule A can include a polypeptide sequence that corresponds to a protein or portion thereof.

FIG. 4B illustrates the supports 88 being disposed in and incubated in each of the three post-translational modifying solutions, kinases a, b, anc c (86a through 86c). The supports 88 including the target molecule A can be incubated for an appropriate amount of time to achieve any post-translational modification that might occur in one or more of the three post-translational modifying solutions 86a through 86c.

FIG. 4C illustrates the separation of the supports 88 from the sample-retaining structures 84a, 84b, and 84c. Target molecule A was not phosphorylated by the kinase a, while target molecule A was phosphorylated (as shown by phosphate 92) by kinases b and c.

Although FIG. 4C depicts the supports 88 being removed from the sample-retaining structures 84a, 84b, and 84c, in another embodiment, the three post-translational modifying solutions 86a through 86c could be removed from the sample-retaining structures 84a, 84b, and 84c. For example, the sample-retaining structures 84a, 84b, and 84c could include a filter so that the three post-translational modifying solutions 86a through 86c could be drained from the sample-retaining structures 84a, 84b, and 84c and the supports 88 could be left remaining in the sample-retaining structures 84a, 84b, and 84c. In the latter embodiment, additional steps (e.g., washing, digestion, and the like) can be conducted in the sample-retaining structures 84a, 84b, and 84c.

As mentioned above, the MALDI plate can include one or more addressable arrays as described above. The MALDI plates may assume a variety of shapes and sizes. The materials of the support(s) are typically substantially chemically and physically stable under conditions employed in the MALDI processing/analysis. For example, the supports may be substantially inert (e.g., substantially chemically and/or physically inert) to any reagents employed (e.g., wash reagents, chemical cleavage or digestion reagents, etc.) during the MALDI processing. It should be noted that the support surface is made to react or can be modified to react with the target molecules so that they bind to the surface. The surface of the support contacting the post-translation modification solution may be modified to be hydrophilic, hydrophobic, and the like. The surface may need to have a layer of material disposed thereon to attach the target polypeptides such as avidin or the like.

As mentioned above, the substrate includes at least one sample-retaining structure on a surface thereof that effectively retains the post-translational modification solution. The substrates may assume a variety of shapes and sizes. The materials of the substrates are typically substantially chemically and physically stable under conditions employed for the sample processing. For example, the substrates may be substantially inert (e.g., substantially chemically and/or physically inert) to the sample contacted thereto and any reagents employed (e.g., wash reagents, chemical cleavage or digestion reagents, etc.) during the sample processing. The surface contacting the post-translational modification solution may be modified to be hydrophilic, hydrophobic, and the like.

The shape of the sample-retaining structure(s) depends, in part, on a variety of factors such as the post-translational modification solution. For example, in an embodiment, the shape is selected such that mating of a sample-retaining structure with an array may be accomplished and/or configured so that the sample-retaining structure is able to accommodate a laser beam directed into the interior thereof (i.e., directed at the sample retained by the sample-retaining structure). As such, the sample-retaining structures may assume a variety of different shapes such that the shapes of these structures range from simple to complex. In many embodiments, the sample-retaining structures are a square, a rectangular, an oblong, an oval or a circular shape, although other shapes are possible as well, such as other geometric shapes, as well as irregular or complex shapes.

The sample-retaining structure(s) can be raised above the surface of the substrate or can be depressions (e.g., wells) in the substrate. Typically, the number of sample-retaining structures present on a substrate ranges from about 1 to 2000 or more; for example, as few as 1, 10, 20, 50, 100, and 500, and as many as about 2500, 3000, 3500, 4000, 4500, and 5000 or more sample-retaining structures may be present on a single substrate. As such, the configuration or pattern of the sample-retaining structures may vary depending on the particular processes being employed, the number of sample-retaining structures present, the size and shape of the sample-retaining structures present, and, in certain embodiments, the size, shape and pattern of the arrays to which the sample-retaining structures are to be joined, and the like.

In general, the dimensions of the sample-retaining structure are such that any fluid retaining structure is able to accommodate a volume of fluid sufficient to perform the sample processing. Typically, the sample-retaining structures have a volume ranging from about 0.1 μL to about 10 μL or more, in certain embodiments from about 0.1 μL to about 5 μL and in certain embodiments from about 0.1 μL to about 2 μL.

Any material having suitable characteristics may be used as the sample-retaining structure material. Suitable sample-retaining structure material may derive from naturally occurring materials, naturally occurring materials that have been synthetically modified, or synthetic materials. Sample-retaining structure materials are generally fluid materials that may be cured to provide a solid fluid retaining structure having suitable characteristics. Selection of the sample-retaining structure material is determined relative to the intended application. Suitable sample-retaining structure materials include, but are not limited to, polymers, elastomers, silicone sealants, urethanes, and polysulfides, latex, acrylic, and the like.

The amount of analyte solution deposited into a fluid retaining structure will vary depending on the analyte, but will typically range from about 1 μL to 50 μL, more usually from about 1 μL to 10 μL and more usually from about 1 μL to 5 μL, where the amount of analyte present may range from about 0.1 picogram (pg) to 1 microgram (μg), usually from about 1 pg to 1 μg, and more usually from about 10 pg to about 1000 μg. The analyte may be deposited into a fluid retaining structure in any convenient manner, e.g., the analyte may be manually deposited, e.g., pipetted, or automatically deposited using a fluid dispensing machine such as a robotic dispensing machine or the like, or transferred via an array.

A mass spectrometry system is an analytical system used for quantitative and qualitative determination of the compounds/components of a material (e.g., chemical mixtures and biological samples). In general, a mass spectrometry system uses an ion source to produce electrically charged particles (e.g., molecular or polyatomic ions) from the material to be analyzed. Once produced, the electrically charged particles are introduced to the mass spectrometer and separated by a mass analyzer based on their respective mass-to-charge ratios. The abundance of the separated electrically charged particles are then detected and a mass spectrum of the material is produced. The mass spectrum is analogous to a fingerprint of the sample material being analyzed. The mass spectrum provides information about the mass-to-charge ratio of a particular compound in a mixture sample and, in some cases, molecular structure of that component in the mixture.

The molecular weight of a compound is often determined by the use of a mass spectrometry system having a single mass analyzer. The mass analyzer may include a quadrupole (Q) mass analyzer, a time-of-flight mass analyzer (TOF-MS), an ion trap mass analyzer (IT-MS), etc. Tandem mass spectrometers (i.e., tandem-MS or MS/MS) are often needed to analyze samples having complicated molecules. Tandem mass analyzers typically include two mass analyzers of the same or different type (e.g., TOF-TOF MS and Q-TOF MS).

In a tandem mass spectrometry analysis, electrically charged particles are transmitted to the first mass analyzer, and an ion of particular interest is selected. The selected ion is transmitted to a dissociation cell where the selected ion is fragmented.

The ionic fragments of the dissociated ion are transmitted to the second mass analyzer for mass analysis. The fragmentation pattern obtained from the second mass analyzer is then analyzed to determine the structure of the corresponding molecule.

Matrix assisted laser desorption/ionization mass spectrometry (MALDI-MS) is of particular importance for the analysis of biological samples, which was introduced by Karas et.al. (Karas, M.; Hillenkamp, F., Anal. Chem. 60, p. 2299, 1988) and Tanaka et.al. (Tanaka, K.; Waki, H.; Ido, Y.; Akita, S.; Yoshida, Y.; Yoshica, T., Rapid Commun. Mass Spectrom. 2, p. 151, 1988). MALDI-MS has also become established as a method of mass spectrometry used to analyze substances such as polypeptides, polynucleotides, proteins, DNA fragments, biopolymers, and other large molecules.

MALDI-MS allows for desorption and ionization of non-volatile samples (e.g., biological samples) from a solid-state phase directly into the gas phase without charring, fragmentation, or chemical degradation. The sample (analyte) is suspended or dissolved in a matrix. Matrices are small organic compounds that are co-crystallized with the analyte.

Laser radiation (i.e., a laser beam) produced by a laser generator, serves as a desorption and ionization source in MALDI-MS. The matrix absorbs the laser energy and causes part of the illuminated sample to vaporize. A rapidly expanding matrix plume carries some of the analyte ions into the vacuum of the mass spectrometer. The matrix molecules absorb most of the incident laser energy minimizing sample damage and ion fragmentation (i.e., soft ionization). Laser generators of various wavelengths (e.g., ultraviolet and infrared) have been used in MALDI-MS.

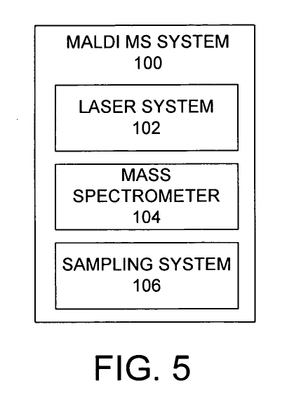

FIG. 5 is a block diagram that illustrates a representative MALDI-MS system 100. The MALDI-MS system 100 includes a laser system 102, a mass spectrometer 104, and a sampling system 106. The sampling system 106 can include a MALDI plate that is to be analyzed. The mass spectrometer 104 includes a mass analyzer/detection system.

The laser system 102 emits laser radiation that impinges the MALDI plate located within the sampling system 106. The sample absorbs the laser energy, which causes part of the laser illuminated portion of the sample to vaporize. The rapidly expanding matrix plume carries some of the analyte ions into the vacuum of an ion optic system. The ions may take the form of a packet of ions (i.e., generated with laser radiation pulse) or a constant stream of ions (i.e., generated with a constant beam of laser radiation). Once the sample molecules are vaporized and ionized, the ions are transferred via the ion optic system into the mass analyzer/detection system. The ions are separated in the mass analyzer/detection system according to their mass-to-charge ratio and detected by a detector based on their relative abundance.

This process can be performed a plurality of times for a particular position upon the sample. Optionally, the sample can be repositioned by moving the MALDI plate in the x-, y-, and/or z-axis to obtain ions from various positions of the sample (e.g., profiling).

The mass analyzer/detection system can include a mass analyzer such as, for example, a time-of-flight (TOF) mass analyzer, an ion trap mass analyzer (IT-MS), a quadrupole (Q) mass analyzer, a magnetic sector mass analyzer, or an ion cyclotron resonance (ICR) mass analyzer. In one embodiment, because it can be used to separate ions having very high masses, the mass analyzer is a TOF mass analyzer.

In addition, the mass analyzer/detection system includes an ion detector. An ion detector is a device for recording the number of ions that are subjected to an arrival time or position in a mass spectrometry system, as is known in the art. Ion detectors can include, for example, a microchannel plate multiplier detector, an electron multiplier detector, or a combination thereof. In addition, the mass analyzer/detection system includes vacuum system components and electronic system components, as are known in the art.

The laser system 102 includes a laser generator and a laser optic system. The laser generator can include a laser generator such as, for example, a Nd:YAG laser having an output wavelength of 1060 nanometers and a harmonic wavelength of 533 nanometers, 353 nanometers and 256 nanometers, a nitrogen laser having an output wavelength of 337 nanometers, or a CO₂ laser having an output wavelength of 10.6 micrometers, a tunable laser, or another laser having a wavelength ranging from ultraviolet wavelengths to infrared wavelengths, as are known in the art. The laser used in a laser desorptionlionization source is usually a pulsed laser with a repetition rate of 1 to 100 Hertz, typically 10 to 20 Hertz. Other laser optic systems, known in the art, typically used in conjunction with laser generators, can be used in the laser system 102.

The sampling system 106 includes the MALDI plate. The sampling system 106 facilitates the movement (i.e., x-y-z directions) of the sampling substrate manually or through the use of a computer system. Sample substrate systems 106 are known in the art and will not be discussed in detail.

### EXAMPLES

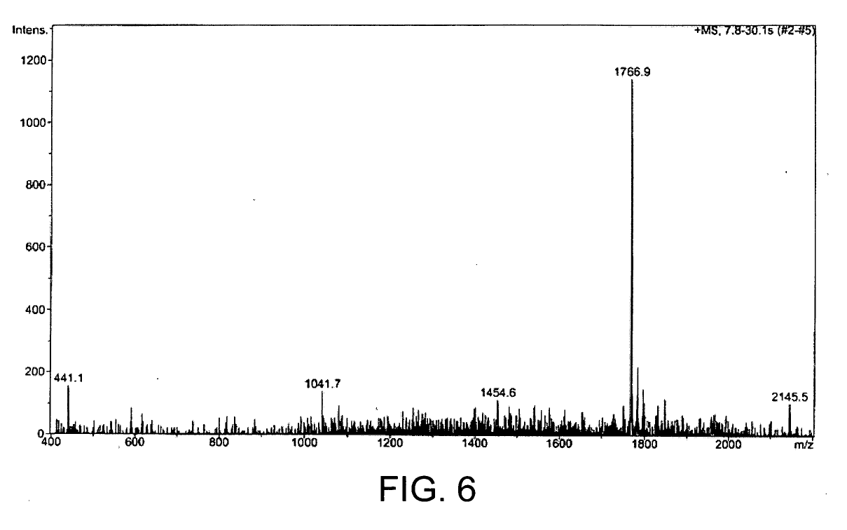

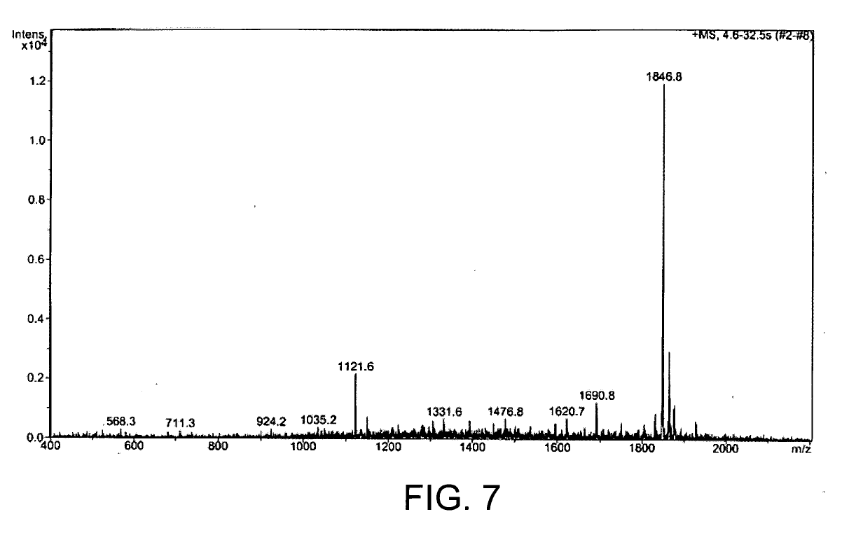

The MALDI spectra (FIGS. 6 and 7) were obtained with an atmospheric pressure laser desorption ionization interface set up with a nitrogen laser (337 nm) with a repetition rate of 20 Hz. The matrix was alpha-cyano 4-hydroxy cinnamic acid. One can clearly see from the two mass spectra that the M+1 ion of the phosphorylated peptide is shifted by a mass of 80 Da, which corresponds to HPO₃ (of the phosphate group). Specifically, the unphosphorylated peptide exhibits a protonated molecular ion at m/z (or mass-to-charge ratio) of 1766.9 amu (atomic mass units) and the phosphorylated peptide exhibits a protonated molecular ion at m/z of 1846.8 amu. The mass difference between those two protonated molecular ions corrresponds exactly to the mass of the phosphate group; thus, it was proved that a phosphorylated peptide could be identified from its MALDI mass spectrum.

FIG. 6 illustrates a MALDI mass spectrum of biotinylated Kir 2.1 peptide (biotin-SEQ ID: 1 (SGSGPRPLRRESEI)-COOH)).

FIG. 7 illustrates a MALDI mass spectrum of biotinylated and phosphorylated Kir2.1 peptide, where the only difference in the sequence in that the serine (third to last amino acid) is phosphorylated (biotin-SEQ ID: 1 (SGSGPRPLRRES(Phosphate)EI)-COOH).

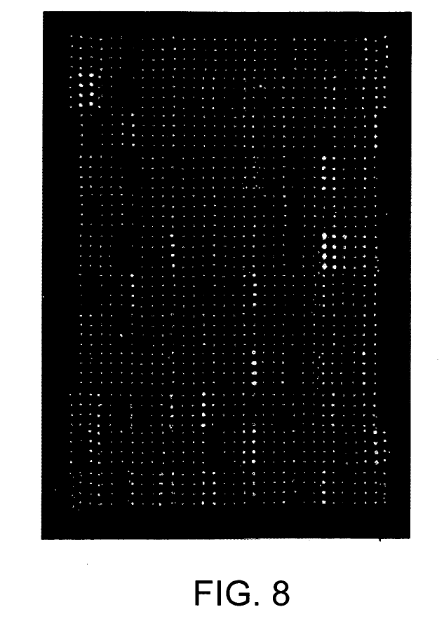

FIG. 8 illustrates a peptide array that contains 181 peptides, each printed in quadruplicate (32×48 spots). The peptide array was made using a thermal ink-jet deposition tool, which deposited each peptide onto a functionalized glass slide. Each spot is approximately 50 μm in diameter and contains approximately 7 picograms of each peptide (a peptide solution at 0.2 mg/mL was used and 35 pL was delivered through the nozzle of the deposition tool). The array contains one peptide known as Histone-H3 (sequence: SEQ ID: 3 (ARTKQTARKSTGGKAPRKQLAGGK)-biotin) in the lower right-hand corner or column 31, rows 45-48) that is phosphorylated by Aurora B kinase.

Using the array to find out whether the kinase is phosphorylating the peptide can be done using the following illustrative procedure:

1. Prepare buffer (tris assay dilution buffer consisting of 500 mM Tris-HCl pH 7.5, 1 mM EGTA, 150 mM dithiothreitol);

2. Prepare magnesium ATP cocktail (75 mM magnesium chloride and 500 uM ATP in 20 mM MOPS pH 7.2, 25 mM beta-glycerophosphate, 5 mM EGTA, 1 mM trisodium vanadate, 1 mM dithiothreitol);

3. Add Aurora B (solution in 50 mM Tris-HCl, 150 mM NaCl, 0.1 mM EGTA and 0.1% beta-mercaptoethanol) and magnesium ATP cocktail to a gasket slide and incubate for 20 min at room temperature;

4. Separate the slides, wash the array and spin it dry in a centrifuge;

5. Attach peptide array (that should now have the phosphorylated Histone H3) to the MALDI plate and add matrix; and

6. Get MALDI mass spectrum.

It should be emphasized that the above-described embodiments of the present disclosure are merely possible examples of implementations, merely set forth for a clear understanding of the principles of the disclosure. Many variations and modifications may be made to the above-described embodiment(s) of the disclosure without departing substantially from the principles of the disclosure. All such modifications and variations are intended to be included herein within the scope of this disclosure and the present disclosure and protected by the following claims.

We claim:

1. A screening method for identifying target molecules capable of being post-translationally modified, comprising:
providing a substrate having a sample-retaining structure disposed on the substrate and a post-translational modifying solution disposed in the retaining structure;
providing a matrix assisted laser desorption/ionization (MALDI) plate having a plurality of target molecules disposed on the MALDI plate, wherein each target molecule has a known addressable position on the MALDI plate;
exposing the plurality of target molecules to the post-translational modifying solution by disposing the MALDI plate onto the substrate and retaining structure so that the plurality of target molecules are disposed into the post-translational modifying solution, wherein the post-translational modifying solution reacts with select target molecules to produce post-translationally modified target molecules; and
analyzing the post-translationally modified target molecules using a matrix assisted laser desorption/ionization mass spectrometry system (MALDI-MS system).

2. The method of claim 1, further comprising:
determining the addressable position of each of the post-translational modified target molecules.

3. The method of claim 1, wherein the post-translational modifying solution includes post-translational modifying molecules comprising kinases, and wherein the post-translational modified target molecules are phosphorylated.

4. The method of claim 1, wherein the post-translational modifying solution includes post-translational modifying molecules comprising a phosphatase, and wherein the post-translational modified target molecules are dephosphorylated.

5. The method of claim 1, wherein the post-translational modifying solution includes post-translational modifying molecules comprising a sulfotransferase, and wherein the post-translational modified target molecules are sulfated.

6. The method of claim 1, wherein the post-translational modifying solution includes post-translational modifying molecules comprising a sulfatase, and wherein the post-translational modified target molecules are desulfated.

7. A screening method for identifying target molecules capable of being post-translationally modified, comprising:
providing a substrate having a plurality of sample-retaining structures, wherein at least two sample-retaining structures have a different post-translational modifying solution disposed in each of the at least two sample-retaining structures;
providing a plurality of supports having at least one target molecule disposed thereon;
disposing a portion of each of the supports into each of the sample-retaining structures, wherein the target molecules are disposed into one of the post-translational modifying solutions in the sample-retaining structures, wherein the post-translational modifying solution reacts with select target molecules to produce post-translational modified target molecules;
separating the supports from the post-translational modifying solutions;
removing one or both of the target molecules or the post-translational modified target molecules from the supports;
disposing one or both of the target molecules or the post-translational modified target molecules onto a matrix assisted laser desorption/ionization (MALDI) plate, wherein each of the target molecules and the post-translational modified target molecules have a known addressable position on the MALDI plate;
operatively associating the MALDI plate with a matrix assisted laser desorption/ionization mass spectrometry system (MALDI-MS system);
illuminating a portion of the MALDI plate with a laser of the MALDI-MS system, wherein one or both of the target molecules or post-translational modified target molecules are ionized; and
collecting mass spectral data of one or more of the ionized target molecules or post-translational modified target molecules.

8. The method of claim 7, further comprising:
determining the addressable position of each of the post-translational modified target molecules.

9. The method of claim 7, wherein the post-translational modifying solutions include post-translational modifying molecules comprising at least two types of kinases, and wherein the post-translational modified target molecules are phosphorylated.

10. The method of claim 7, wherein the post-translational modifying solutions include post-translational modifying molecules comprising at least two types of phosphatases, and wherein the post-translational modified target molecules are dephosphorylated.

11. The method of claim 7, wherein the post-translational modifying solutions include post-translational modifying molecules comprising at least two types of sulfotransferases, and wherein the post-translational modified target molecules are sulfated.

12. The method of claim 7, wherein the post-translational modifying solutions include post-translational modifying molecules comprising at least two types of sulfatases, and wherein the post-translational modified target molecules are desulfated.
